(19) United States
(12) Patent Application Publication
(10) Pub. No.: US 2005/0004429 A1
(43) Pub. Date: Jan. 6, 2005

(54) Chilled sexual aid

(76) Inventor: Stephen Tracanna, Norristown, PA (US)

Correspondence Address: RATNERPRESTIA, P O BOX 980, VALLEY FORGE, PA, 19482-0980, US

(21) Appl. No.: 10/611,126

(22) Filed: Jul. 1, 2003

(51) Int. Cl. A61F 5/00

(52) U.S. Cl. 600/38

(57) ABSTRACT

A personal sexual aid and a two person sexual aid adapted for chilling. The personal sexual aid a hollow, substantially cylindrical member having a thermal insulating property and freeze gel material having a large thermal mass. The hollow, substantially cylindrical member includes: a side surface; a sealed, substantially dome-shaped front end; and a rear end. The freeze gel material fills the hollow, substantially cylindrical member. The two-person sexual aid includes two hollow, substantially cylindrical members coupled at their rear ends by a coupling section.

### FIELD OF THE INVENTION

This invention is in the field of sexual aids, and specifically relates to a personal sexual aid that may be chilled and inserted in bodily cavities for entertainment purposes.

### BACKGROUND OF THE INVENTION

Among items commercially available for enhancing sexual functioning are dildos, vaginal exercise bars, and prostate stimulators. These items are made of a variety of materials, including latex rubber, silicone, plastic, wood, glass, and metal, and are generally elongate and cylindrical in shape. Their length, width, and shape vary within anatomically prescribed limits. They may incorporate design features imitative of a wide range of natural, mechanical, and imaginary objects, e.g., vegetables such as a cucumber or ear of corn, a rocket ship, a dolphin, or the human penis or finger; or the design may be more abstract. The distal end may have a handle (which is not inserted) or other means for attachment to a stationary or moving object, or to the surface of a partner's body. In use they are inserted into the vagina or anus, either by an individual or by a partner, using suitable lubrication as required. They provide stimulation from friction upon manipulation of the object, or by pressure from the distension of the cavity due to the volume of the object. Some objects are designed to create additional sensory effects in the user through the production of heat, fluid discharge, or low voltage electrical stimulation or vibration. Some objects create diffuse sensations in the user, while others are designed to target specific areas of the body cavity, such as the prostate gland in men or the paraurethral gland of the urethral sponge of the clitoris in women (also called the Grafenberg spot or G spot), or they may have surface texture, such as raised bumps or ridges which enhance local pressures upon manipulation of the object.

Dildos and the related personal sexual aids described above are used by individuals of either sex, by transgendered (sex-changed) persons, and by couples (both heterosexual and homosexual) to give sexual pleasure to each other, either in lieu of or in addition to vaginal/penile or other modes of sexual congress. Hence, such couples may be comprised of two women, a man and a woman, two men, two transgendered persons, or a transgendered person and either a man or a woman. Double-ended dildos are also marketed, for simultaneous use by couples. In addition to their use for pleasure, the use of items such as vaginal exercise bars and prostate stimulators may be recommended by physicians and other health professionals for the purpose of developing, strengthening, tightening, or otherwise enhancing the function of muscles, glands, and other organs surrounding the vagina and/or urethra and/or anus. Conditions in which such use may be recommended include post-partum vaginal recovery, prostatitis, multiple sclerosis, anorgasmia, and urinary incontinence. The use of these objects may also be recommended for purposes such as contraception, avoidance of disease (including AIDS), disability, erectile dysfunction, and temporary fatigue.

### SUMMARY OF THE INVENTION

One embodiment of the present invention is an exemplary personal sexual aid adapted for chilling. The personal sexual aid a hollow, substantially cylindrical member having a thermal insulating property and freeze gel material having a large thermal mass. The hollow, substantially cylindrical member includes: a side surface; a sealed, substantially dome-shaped front end; and a rear end. The freeze gel material fills the hollow, substantially cylindrical member.

Another embodiment of the present invention is an exemplary chilled two-person sexual aid. The chilled two-person sexual aid includes two hollow, substantially cylindrical members having thermal insulating properties, freeze gel material having a large thermal mass, and a center coupling section. Each hollow, substantially cylindrical member includes: a side surface; a sealed, substantially dome-shaped front end; and a rear end. The freeze gel material fills the hollow, substantially cylindrical members. The center coupling section couples the rear ends of the two hollow, substantially cylindrical members

An additional embodiment of the present invention is an alternative exemplary personal sexual aid adapted for chilling. The personal sexual aid includes a partially hollow and partially solid, substantially cylindrical member having a thermal insulating property; and freeze gel material having a large thermal mass. The solid portion of the substantially cylindrical member forms a substantially dome-shaped front end of the personal sexual aid. The freeze gel material fills the hollow portion of the substantially cylindrical member.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a side plan drawing of an exemplary chilled sexual aid according to the present invention.

FIG. 2 is a side plan drawing of an alternative exemplary chilled sexual aid according to the present invention.

FIG. 3 is a side plan drawing of an exemplary double ended chilled sexual aid according to the present invention.

FIG. 4 is a side plan drawing of an alternative exemplary double ended chilled sexual aid according to the present invention.

FIG. 5 is a side plan drawing of an exemplary textured chilled sexual aid according to the present invention.

FIG. 6 is a side plan drawing of an exemplary vibrating chilled sexual aid according to the present invention.

FIG. 7 is a side plan drawing of an exemplary chilled sexual aid including a suction cup for mounting according to the present invention.

### DETAILED DESCRIPTION

The present invention involves a personal sexual aid, which includes a freeze gel that may be desirably chilled to provide a pleasurable and/or soothing touch during use. The cooling sensation of an exemplary personal sexual aid according to the present invention may be used either erotically to arouse the person on whom it is used, or therapeutically to sooth sore tissues, particularly tissues within bodily orifices. The desired operating temperature of an exemplary personal sexual aid according to the present invention may vary from one individual to the next and one use to the next, but it is desirably less than room temperature (approximately 20-30° C.) and greater than a temperature that may cause frostbite (<0° C.). It is desirable in both erotic and therapeutic uses for the cooling properties of the exemplary personal sexual aid to be able to maintained at a relatively constantly near, or at least slowly rising from near, the desired operating temperature over an extended period of time.

Before use, the exemplary personal sexual aid is desirably chilled, using a refrigerator, freezer, ice bath, or other standard means. The initial operating temperature may be controlled by that temperature the freeze gel is chilled to before use, either by controlling the temperature of the chilling means or the time that the exemplary personal sexual aid is stored in the chilling means. If the exemplary personal sexual aid is found to be undesirably cold it may be warmed until the desired operating temperature is achieved.

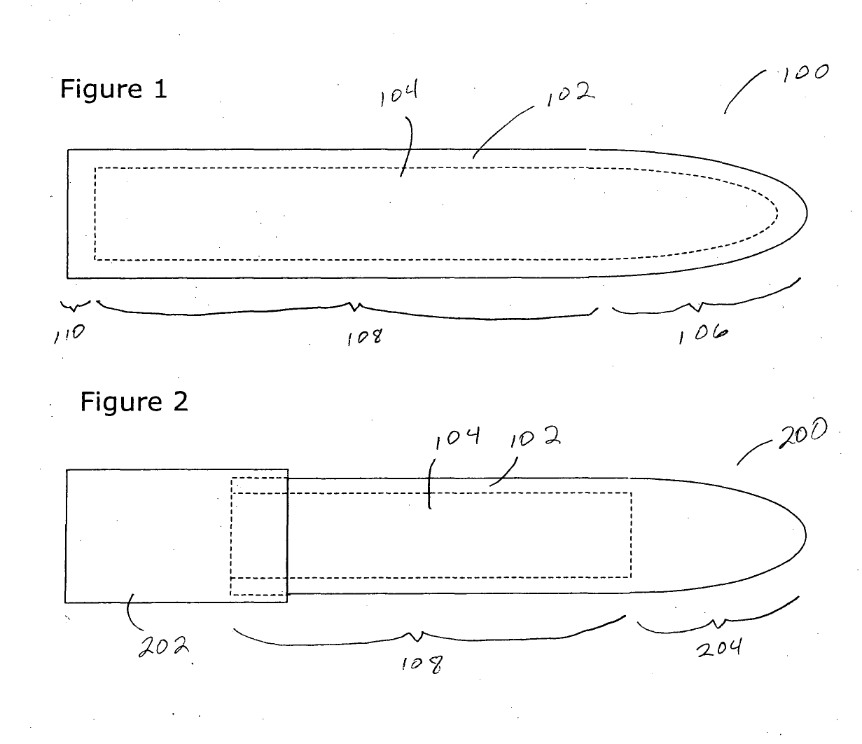

One exemplary embodiment of the present invention, illustrated in FIG. 1, is exemplary single piece personal sexual aid 100. This exemplary embodiment includes hollow, substantially cylindrical member 102 and freeze gel 104. Desirably, freeze gel 104 is composed of a gel material which has a large thermal mass to help maintain the device at the desired operating temperature, by requiring a large amount of heat to increase the temperature of the freeze gel. These gel materials include carboxy methyl cellulose gels, hydrogels such as agar hydrogels and κ-carrageenan hydrogels, gelatin gels, and polyvinyl alcohol gels. The thermal mass of freeze gel 104 is affected by the volume of gel material included, as well as the type of gel material. Also, the amount of heat that may be absorbed during use is affected by the initial temperature of freeze gel in addition to the thermal mass. It is noted that, the gel material may also desirably be selected to either remain flexible or to become solid and rigid when chilled for use.

Hollow, substantially cylindrical member 102 forms the body of the exemplary device and additionally provides a layer of thermal insulation between freeze gel 104 and the, person(s) using the device. Hollow, substantially cylindrical member 102 may be formed of one or more material commonly used to form sexual aids, including latex rubber, silicone, glass, acrylic, cyberskin, and jelly vinyl. The thermally insulating property of hollow, substantially cylindrical member 102 allows a thermal gradient to occur in its material. Thus, the outer surface of hollow, substantially cylindrical member 102 may be maintained at a desirable, pleasurable and/or soothing, operating temperature, while the freeze gel may desirably have a temperature well below any temperature that is pleasurable and/or soothing. This thermally insulating property of hollow, substantially cylindrical member 102 also slows heat transfer from the body of the user(s) to freeze gel 104. The amount of thermal insulation provided by hollow, substantially cylindrical member 102 is affected by the thickness of the material as well as type of material used to form it.

Desirably, the materials and radial dimensions of hollow, substantially cylindrical member 102 and freeze gel 104 may be selected so the outer surface of exemplary personal sexual aid 100 has a desirable operating temperature when the freeze gel material is chilled to between approximately −5 to 5° C. Also, it is desirable for the large thermal mass of freeze gel 104 and the thermal insulating property of hollow, substantially cylindrical member 102 to be selected such that the operating temperature may be maintained in a range near (within approximately 10° C.) for approximately an hour after the freeze gel material is chilled. The total radial dimension of exemplary personal sexual aid 100 is somewhat constrained by its desired use for insertion into bodily orifices, such as the anus and the vagina. Also, although hollow, substantially cylindrical member 102 is shown as a straight substantially phallic shape in FIGS. 1-7, it is understood that this member may be curved longitudinally as well.

In addition to its thermal insulating properties, the material of hollow, substantially cylindrical member 102 may desirably be chosen so that the material remains flexible when chilled to the desired operating temperature. This exemplary embodiment of the chilled personal sexual aid may be filled with a gel material that also remains flexible when chilled, or may be filled with a gel material that hardens when chilled to provide rigidity for the device while the flexible material of outer hollow, substantially cylindrical member 102 allows the device to have a softer feel. Alternatively, hollow, substantially cylindrical member 102 may be formed of a material that is solid and rigid at the desired operating temperature and/or room temperature as well. It is noted that an exemplary chilled personal sexual aids in which both hollow, substantially cylindrical member 102 and freeze gel 104 are flexible at room temperature, but one or both are solid and rigid when chilled, may be held in a curved shape during chilling and desirably maintain this curve shape during use.

Exemplary hollow, substantially cylindrical member 102 may be visualized of as including three sections: side surfacelO8; sealed, substantially dome-shaped front end 106; and rear end 110, which is sealed to contain the gel material of freeze gel 104. Rear end 110 may extend longitudinally several inches past freeze gel 104 to provide a hand hold that may be desirably warmer than side surfacelO8 and sealed, substantially dome-shaped front end 106 during use.

FIG. 2 illustrates alternative exemplary chilled personal sexual aid 200. In this alternative embodiment, hollow, substantially cylindrical member 102 includes solid substantially dome-shaped front end 204, and rear end 110 has been replaced by separate end cap 202. Solid substantially dome-shaped front end 204 may be formed of the same material as the rest of hollow, substantially cylindrical member 102 or may include an outer layer formed of the cylindrical member material and an inner core of material that is solid and rigid when chilled. Desirably, end cap 202 may be formed of one or more material commonly used to form sexual aids, including latex rubber, silicone, glass, acrylic, cyberskin, and jelly vinyl. It may be desirable for end cap 202 to be solid and rigid at room temperature and/or to provide greater thermal insulation than hollow, substantially cylindrical member 102 may. Alternatively, end cap 202 and hollow, substantially cylindrical member 102 may formed of the same material(s).

The two piece construction of the outer portion of exemplary chilled personal sexual aid 200 may simplify manufacture. Hollow, substantially cylindrical member 102 may be filled with freeze gel 104 and then end cap 202 may be coupled to side surface 108 of hollow, substantially cylindrical member 102 to seal the freeze gel within. End cap 202 may be coupled to side surface 108 of hollow, substantially cylindrical member 102 by press fitting end cap 202 over side surface 108 of hollow, substantially cylindrical member 102, as shown in FIG. 2, or these two components may be threaded and screwed together. Adhesive may also be used to help ensure that the freeze gel is sealed inside of the device, or exemplary chilled personal sexual aid 200 may be heated to seal the two members. Adhesive and or heating may be particularly desirable if either end cap 202 or side surface 108 of hollow, substantially cylindrical member 102 is flexible at room temperature. It is also noted that end cap 202 may be designed to fit within, rather than over, side surface 108 of hollow, substantially cylindrical member 102

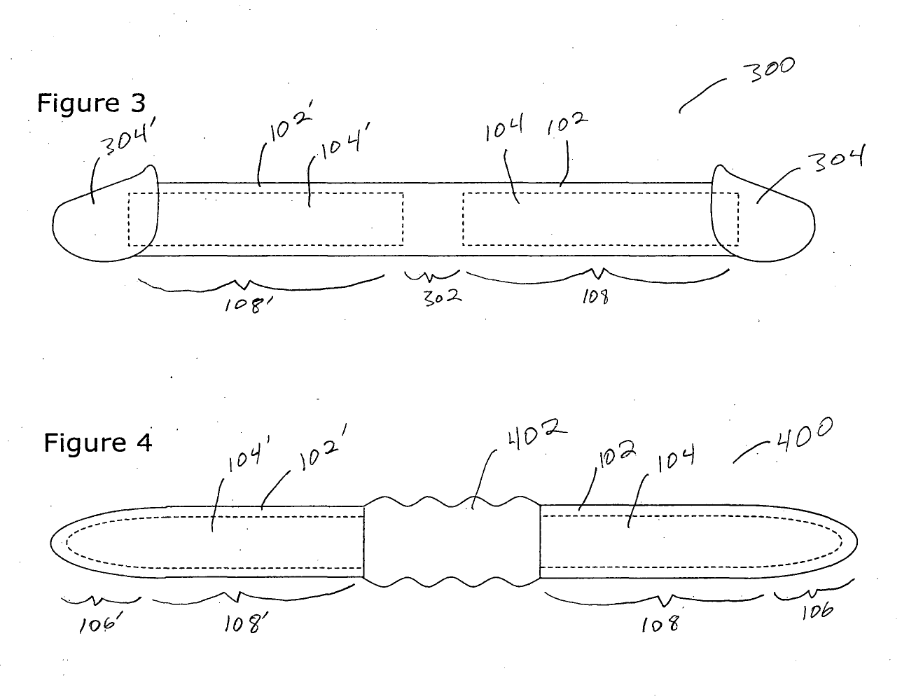

FIG. 3 illustrates exemplary chilled two-person sexual aid 300. Exemplary chilled two-person sexual aid 300 includes two hollow, substantially cylindrical members 102 and 102′, having side portions 108 and 108′, filled with freeze gels 104 and 104′, respectively. Although hollow, substantially cylindrical members 102 and 102′ are shown to be similarly size and shaped, one skilled in the art may understand that this is not necessary.

Hollow, substantially cylindrical members 102 and 102′ are coupled at their rear ends by center coupling section 302. This coupling section may a solid, monolithic section formed of the same materials as hollow, substantially cylindrical members 102 and 102′, as shown in FIG. 3, or may be a separate double ended end cap similar to end cap 202 of FIG. 2. Alternatively, center coupling section 302 may be hollow so that the exemplary chilled two-person sexual aid include only one freeze gel chamber. It is also noted that center coupling section 302 may desirably be flexible when chilled, and or may be curved to provide an angle between the two hollow, substantially cylindrical members.

The sealed, substantially dome-shaped front end portions of hollow, substantially cylindrical members 102 and 102′ are shown in FIG. 3 as phallic heads 304 and 304′, respectively, shaped to simulate the head of a penis.

FIG. 4 illustrates alternative exemplary chilled two-person sexual aid 400. In this alternative embodiment the center coupling section includes handle portion 402 designed to ergonomically fit a hand. This exemplary embodiment also shows sealed, substantially dome-shaped front end portions 106 and 106′ of hollow, substantially cylindrical members 102 and 102′, respectively.

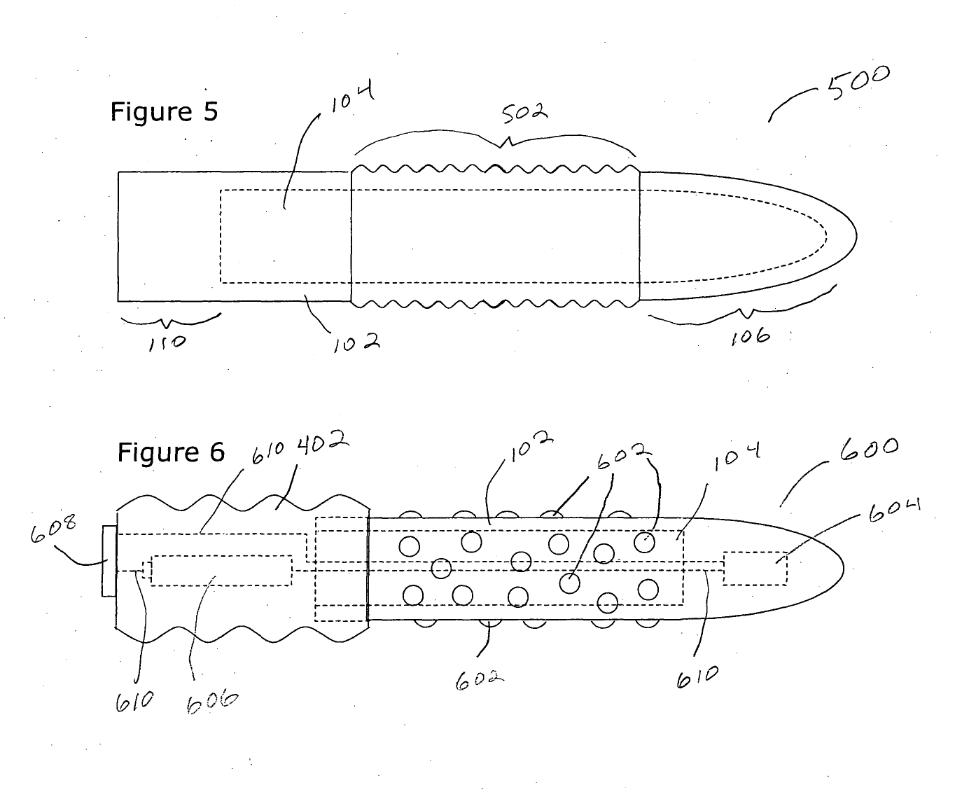

FIG. 5 illustrate another exemplary embodiment of a chilled personal sexual aid 500 accord to the present invention. Exemplary chilled personal sexual aid 500 includes ridged portion 502 along the side surface of hollow, substantially cylindrical member 102. Although ridged portion 502 is shown in FIG. 5 as a periodic series of ridges, it is noted that other ridge designs, such as vein-like bulges to create a more realistic phallic shape, may be used as well. Also rear end 110 of hollow, substantially cylindrical member 102 is shown as somewhat extended in this exemplary embodiment.

FIG. 6 illustrates exemplary vibrating chilled personal sexual aid 600. This exemplary device includes vibrating motor 604 in the sealed, substantially dome-shaped front end of the hollow, substantially cylindrical member 102. This vibrating motor is electrically coupled to battery 606 and control 608 by wires 610. Control 608 desirably includes an on/off switch and may also include a variable resistor for speed control of vibrating motor 604. Although battery 606 and control 608 are shown as part of the end cap portion of exemplary vibrating chilled personal sexual aid 600, it is noted that wires 610 may be extend beyond the rear end of the device to a separate power/control unit. Battery 606 and control 608 may be housed in this separate power/control unit.

It is noted that the end cap of exemplary vibrating chilled personal sexual aid 600 also includes handle portion 402, and the side surface of hollow, substantially cylindrical member 102 includes bumps 602. These bumps may desirably be formed of the same material(s) as hollow, substantially cylindrical member 102.

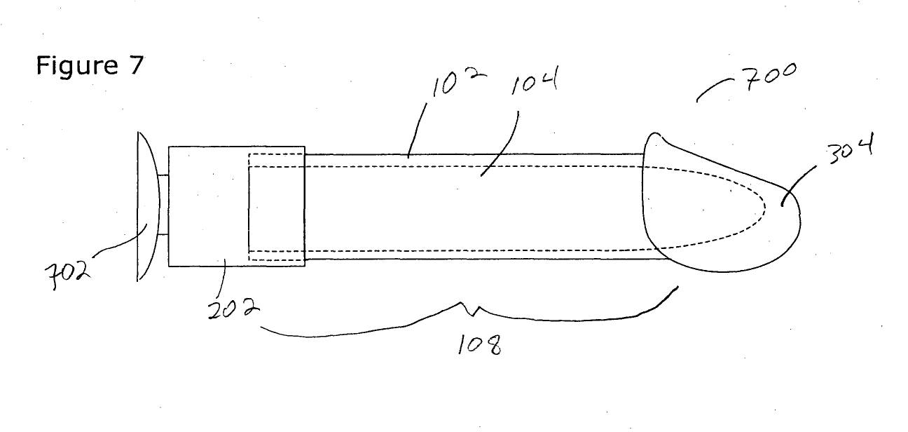

FIG. 7 illustrate additional exemplary chilled personal sexual aid 700. This exemplary device includes suction cup 702 coupled to end cap 202 so that the device may be mounted to a surface such as a wall or a table. Other mounting means such as straps may alternatively be used. Exemplary chilled personal sexual aid 700 also includes phallic head 304. Phallic head 304 may be substantially filled with freeze gel 104, as shown in FIG. 7, or may be solid, as shown in FIG. 3.

While the invention has been described with respect to particular embodiments, those of ordinary skill in the art will appreciate variations in structure and substitutions of materials that are within the scope and spirit of the invention as defined by the appended claims. In particular, features of various exemplary embodiments may be used in combination with features of other exemplary embodiments.

1. A personal sexual aid adapted for chilling, comprising:
a hollow, substantially cylindrical member having a thermal insulating property and including;
a side surface;
a sealed, substantially dome-shaped front end; and
a rear end; and
freeze gel material filling the hollow, substantially cylindrical member and having a large thermal mass.

2. The chilled personal sexual aid of claim 1, wherein the side surface of the hollow, substantially cylindrical member includes at least one of:
a plurality of bumps; and
a plurality of ridges.

3. The chilled personal sexual aid of claim 1, wherein the sealed, substantially dome-shaped front end of the hollow, substantially cylindrical member is solid and rigid when chilled.

4. The chilled personal sexual aid of claim 1, wherein the sealed, substantially dome-shaped front end of the hollow, substantially cylindrical member is shaped to simulate the head of a penis.

5. The chilled personal sexual aid of claim 1, wherein the sealed, substantially dome-shaped front end of the hollow, substantially cylindrical member includes a vibrating device.

6. The chilled personal sexual aid of claim 1, wherein the hollow, substantially cylindrical member is formed of at least one of:
latex rubber;
silicone;
acrylic;
glass;
cyberskin; and
jelly vinyl.

7. The chilled personal sexual aid of claim 1, wherein the freeze gel material and the hollow, substantially cylindrical member are flexible when chilled.

8. The chilled personal sexual aid of claim 1, wherein the hollow, substantially cylindrical member is solid and rigid when chilled.

9. The chilled personal sexual aid of claim 1, wherein the freeze gel material is solid and rigid when chilled.

10. The chilled personal sexual aid of claim 1, wherein the freeze gel material is formed of at least one of:
a carboxy methyl cellulose gel;
an agar hydrogel;
a κ-carrageenan hydrogel;
a gelatin gel; and
a polyvinyl alcohol gel.

11. The chilled personal sexual aid of claim 1, further comprising an end cap covering the rear end of the hollow, substantially cylindrical member.

12. The chilled personal sexual aid of claim 11, wherein the end cap is rigid.

13. The chilled personal sexual aid of claim 11, wherein the end cap includes a handle portion.

14. The chilled personal sexual aid of claim 11, wherein the end cap includes a suction cup.

15. The chilled personal sexual aid of claim 1, wherein:
the freeze gel material is chilled to between approximately −5 to 5°C; and
the large thermal mass of the freeze gel material and the thermal insulating property of the hollow, substantially cylindrical member maintain the side surface of the hollow, substantially cylindrical member at a substantially constant operating temperature for approximately an hour after the freeze gel material is chilled, the operating temperature being greater than 0° C.

16. The chilled personal sexual aid of claim 1, wherein the hollow, substantially cylindrical member is adapted for insertion into a bodily orifice.

17. A chilled two-person sexual aid, comprising:
two hollow, substantially cylindrical members having thermal insulating properties, each hollow, substantially cylindrical member including;
a side surface;
a sealed, substantially dome-shaped front end; and
a rear end;
freeze gel material filling the hollow, substantially cylindrical members and having a large thermal mass; and
a center coupling section coupling the rear ends of the two hollow, substantially cylindrical members.

18. The chilled personal sexual aid of claim 17, wherein the center coupling section includes a handle portion.

19. The chilled personal sexual aid of claim 17, wherein the center coupling section is flexible when chilled.

20. The chilled personal sexual aid of claim 17, wherein the center coupling section is rigid.

21. The chilled personal sexual aid of claim 17, wherein each hollow, substantially cylindrical member is adapted for insertion into a bodily orifice.

22. A personal sexual aid adapted for chilling, comprising:
a partially hollow and partially solid, substantially cylindrical member having a thermal insulating property; and
freeze gel material filling the hollow portion of the substantially cylindrical member having a large thermal mass;
wherein the solid portion of the substantially cylindrical member forms a substantially dome-shaped front end of the personal sexual aid.
